(19) United States
(12) Patent Application Publication
(10) Pub. No.: US 2015/0064689 A1
(43) Pub. Date: Mar. 5, 2015

(54) SYSTEM AND METHOD FOR ANALYZING TRANSMISSIBILITY OF INFLUENZA

(71) Applicant: The Charles Stark Draper Laboratory, Inc., Cambridge, MA (US)

(72) Inventor: James Comolli, Boxborough, MA (US)

(21) Appl. No.: 14/013,552

(22) Filed: Aug. 29, 2013

(51) Int. Cl. C12Q 1/70

(52) U.S. Cl. CPC C12Q 1/701
USPC 435/5; 435/287.1

(57) ABSTRACT

The present disclosure provides systems and methods for determining the transmissibility of a pathogen. The system described herein includes a flow device with a plurality of cell culture chambers. Cells, such as primary human tracheal or bronchial epithelial cells, are cultured within the flow device. Cells within a first cell culture chamber are infected with the pathogen. The pathogen's transmissibility is determined by flowing a gas through the flow device such that the gas flows over the infected cells and then over uninfected cells in a second cell culture chamber of the device. By quantifying the amount of virus present in the cells of the second cell culture chamber after a predetermined amount of time, the transmissibility of the pathogen is determined.

### BACKGROUND OF THE DISCLOSURE

Some of the most dangerous pathogens are capable of being aerosolized and transmitted through the air. Presently, the transmissibility of a pathogen is evaluated experimentally in animal models by placing an infected animal in close proximity to an uninfected animal and monitoring the health of the uninfected animal. Such experiments are costly and difficult to repeat. Furthermore, due to species-specific differences, the animal models may not accurately predict the transmissibility seen in human subjects.

### SUMMARY OF THE DISCLOSURE

The present disclosure provides systems and methods for determining the transmissibility of a pathogen. The system described herein includes a flow device with a plurality of chambers. Cells, such as those derived from the tracheal, bronchial, or alveolar tissues, are cultured within the chambers of the flow device. Cells within a first chamber are infected with a pathogen. The pathogen's transmissibility is determined by flowing a gas through the flow device such that the gas flows over the infected cells in a manner that could transport the pathogen to a second chamber of the device. The second chamber contains uninfected cells. By quantifying the amount of virus transferred to the cells of the second cell culture chamber after a predetermined amount of time, the transmissibility of the pathogen is determined.

According to one aspect of the disclosure, a method of determining the transmissibility of a pathogen includes disposing at least one infected cell in a first cell chamber disposed toward a proximal end of a gas flow channel. The method also includes disposing at least one uninfected cell in a second cell chamber disposed toward a distal end of the gas flow channel. The second cell chamber is separated from the first cell chamber by a barrier, either physical or spatial. The method also includes flowing gas through the gas flow channel such that the gas flows across the at least one infected cell and then flows across the at least one uninfected cell and then determining an infection rate of the uninfected cells.

In certain implementations, the gas is flowed through the gas flow channel in a pulsatile manner. In some implementations, the gas is flowed through the gas flow channel in a flow pattern that simulates a human cough. In some implementations, the method also includes incubating the at least one uninfected cell for a predetermined amount of time before determining the infection rate of the previously uninfected cells. The infection rate is determined by measuring the pathogen load of the at least one uninfected cell.

In some implementations, the method also includes introducing a fluorescent protein into the at least one uninfected cell. The fluorescent protein fluoresces when the at least one uninfected cell is infected by a pathogen from the at least one infected cells. In certain implementations, determining the infection rate of the at least one uninfected cells includes measuring a fluorescence level.

In some implementations, the pathogen is a virus, such as an influenza virus. In yet other implementations, an airway fluid is applied to the barrier space and at least one condition in the gas flow channel is measured. The conditions measured are one of a temperature, a pressure, a flow rate, and a humidity.

According to another aspect of the disclosure, a system for determining the transmissibility of a pathogen includes a gas flow channel having a proximal end and a distal end. The gas flow channel further includes a first cell chamber disposed toward the proximal end of the gas flow channel. The first cell chamber is configured to supply nutrients to a basolateral surface of at least one first cell. The gas flow channel also includes a second cell chamber disposed toward the distal end of the gas flow channel. The second cell chamber is also configured to supply nutrients to a basolateral surface of at least one second cell. A barrier space between the first cell chamber and the second cell chamber is also included in the gas flow channel. The barrier space is configured to allow an aerosolized particle to pass from the first cell chamber to the second cell chamber. The system also includes a gas pump configured to flow gas through the gas flow channel at a controlled flow rate and an incubator configured to maintain a controlled atmospheric condition within the gas flow channel.

In some implementations, the gas pump is configured to flow gas through the gas flow channel in pattern that simulates a cough.

According to yet another aspect of the disclosure, a microfluidic flow device includes a gas flow channel having a proximal end and a distal end. The gas flow channel further includes a first cell chamber disposed toward the proximal end of the gas flow channel. The first cell chamber is configured to supply nutrients to a basolateral surface of at least one first cell. The gas flow channel also includes a second cell chamber disposed toward the distal end of the gas flow channel. The second cell chamber is also configured to supply nutrients to a basolateral surface of at least one second cell. The gas flow channel further includes a barrier space between the first cell chamber and the second cell chamber. The barrier space is configured to allow an aerosolized particle to pass from the first cell chamber to the second cell chamber.

In some implementations, the first cell chamber and the second cell chamber include a permeable membrane disposed atop cellular wells. In certain implementations, the barrier space is linear or non-linear. In other implementations, the barrier space is coated with an airway fluid such as a mucous.

In yet other implementations, the gas flow channel is configured to induce a predetermined shear force on the at least one first cell and the at least one second cell.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 illustrates a system for determining the transmissibility of a pathogen, in accordance with an implementation of the present disclosure;

FIG. 2 illustrates a cross sectional view of a flow channel of the system of FIG. 1, in accordance with an implementation of the present disclosure;

FIGS. 3A and 3B illustrate top views of the flow channels of FIG. 1, in accordance with an implementation of the present disclosure;

FIGS. 4A-4D illustrate gas flow patterns suitable for use in the system shown in FIG. 1, in accordance with an implementation of the present disclosure; and

FIG. 5 illustrates a method for determining the transmissibility of a pathogen with the system of FIG. 1, in accordance with an implementation of the present disclosure.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

Pathogens (e.g., viruses and/or bacteria) are often transmitted person to person. The systems and methods described herein enable the assessment of the transmissibility of pathogens, such as the human-adapted influenza virus. The disclosed systems and methods enable the transmissibility of pathogens to be tested under repeatable, laboratory conditions.

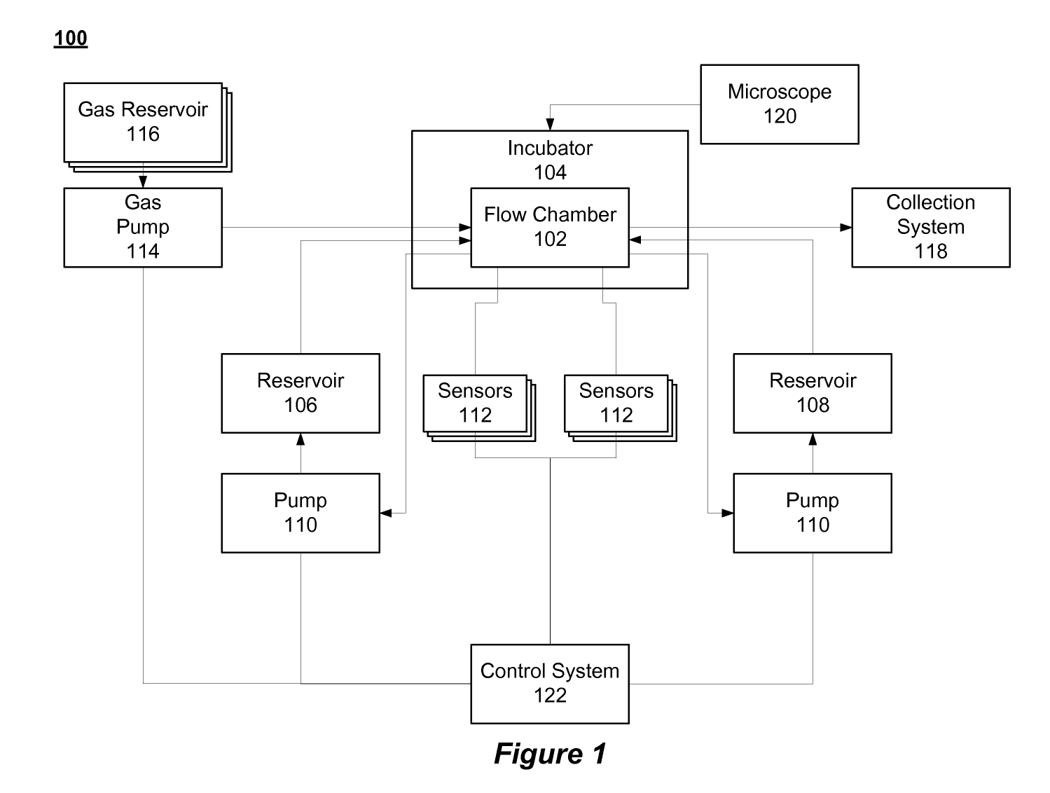

FIG. 1 illustrates one implementation of a system 100 for analyzing the transmissibility of pathogens. The system 100 includes a flow chamber 102 that is housed within an incubator 104. The system 100 also includes a first reservoir 106 and a second reservoir 108. Fluids from the first reservoir 106 and second reservoir 108 are supplied to the flow chamber 102 by pumps 110. A plurality of sensors 112 monitor various parameters in the flow chamber 102. The system 100 further includes a gas pump 114, which drives gas from the plurality of gas reservoirs 116 through the flow chamber 102. A collection system 118 collects the gas and/or fluid as it exits the flow chamber 102. The system 100 also includes a microscope 120 for viewing the cell cultures within the flow chamber 102. The various components of the system 100 are controlled and/or monitored by the control system 122.

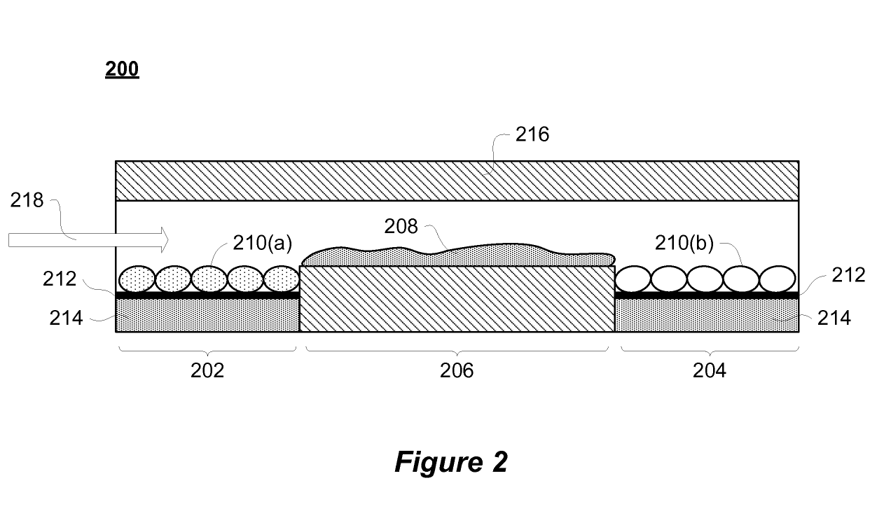

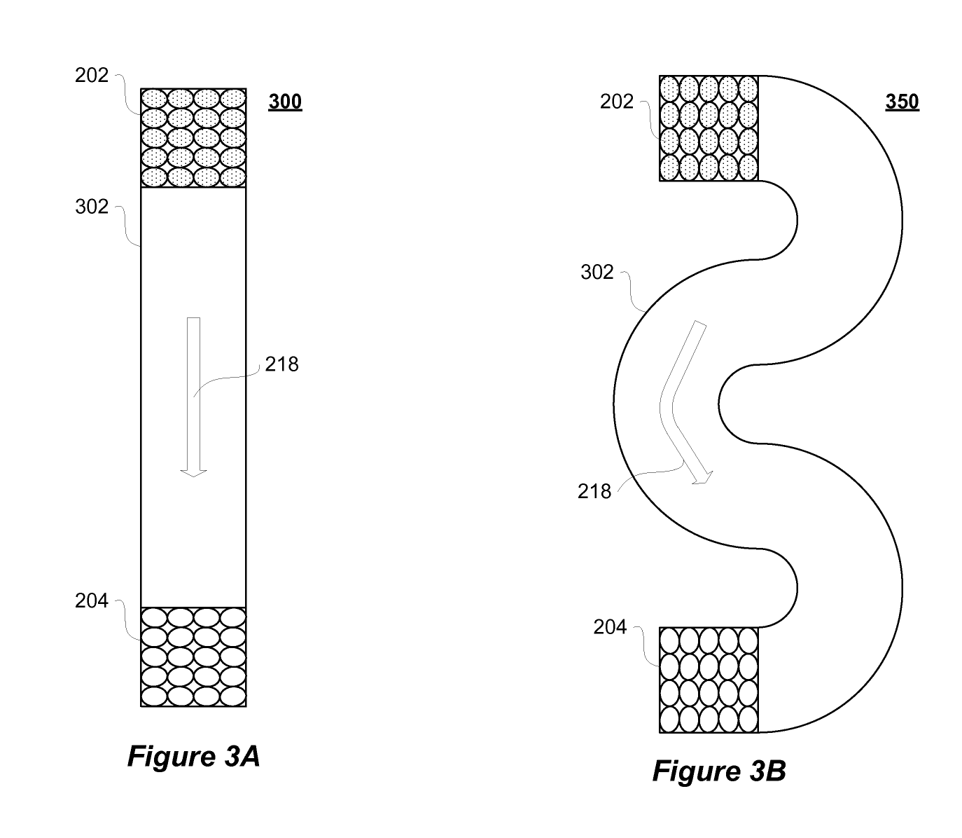

As illustrated, system 100 includes a flow chamber 102. The flow chamber 102 is described in greater detail in relation to FIGS. 2 and 3. Briefly, the flow chamber 102 includes at least one flow channel. Each flow channel includes a first and a second cell culturing area. The cell culturing areas are separated by a barrier space. In some implementations, the cells cultured in the first cell culture are infected with a pathogen and the cells cultured in the second cell culture area are uninfected by the pathogen. The transmissibility of the pathogen is tested by flowing a gas through the flow chamber 102. The transmissibility (including the rate of transmissibility) is determined by quantifying the amount of pathogen present in the cells of the second cell culture after a predetermined amount of time.

The flow chamber 102 is housed within an incubator 104. The incubator 104 maintains an environment within the flow chamber 102 that is conducive for the culturing of the cells. In some implementations, the incubator 104 controls and/or maintains a predetermined temperature, humidity, carbon dioxide level, oxygen level, or any combination thereof. For example, the incubator 104 may be configurable to maintain conditions within the flow chamber 102 that mimic conditions within the human respiratory system, such as a temperature between about 32° C. and about 37° C. with a humidity between about 40% and about 60%. In some implementations, the incubator 104 maintains a 5% carbon dioxide environment within the flow chamber 102. The incubator 104 also includes a plurality of access ports (not illustrated). The ports allow sensor connections and flow lines to pass from the outside environment to the interior of the incubator 104 without affecting the controlled environment within the incubator 104. In some implementations, the incubator 104 is coupled inline with the flow chamber 102 such that the incubator 104 conditions the gas entering the flow chamber 102 as described herein. For example, as gas flows from the gas pump 114 to the flow chamber 102, an inline incubator 104 can heat and oxygenate the gas before it reaches the flow chamber 102.

Also as illustrated, the system 100 includes fluid reservoirs 106 and 108. In some implementations, the fluid reservoirs 106 and 108 store growth medium and/or test agents that are passed to the cell culturing areas via the pumps 110. The fluids supplied to each of the cell culturing areas are distinct from one another (i.e., are stored in separate reservoirs) such that a pathogen may not be passed from the first cell culturing area to the second cell culturing area via the fluid in the reservoirs 106 and 108. In some implementations, the growth medium (or other fluids) stored in the fluid reservoirs 106 and 108 support cell growth, viability, and metabolism. For example, the fluids may include generic growth mediums, Eagle's minimal essential medium and derivatives, specific growth and/or differentiation medium for mammalian primary airway cells, phosphate buffered saline, horse serum, fetal calf serum, buffers or any combination thereof. In some implementations, the test agents include antibiotics such as penicillin, antifungals, antivirals, antimicrobial compounds, additional cells (e.g., immune cells), amino acids, varied energy sources, vitamins, growth factors, trace elements (e.g., metals), or any combination thereof.

The pumps 110 control the flow of fluid from the reservoirs 106 and 108 into and out of the cell culturing areas of the flow chamber 102. In some implementations, the pumps continuously flow fluid into the first and/or second cell culturing areas. In another example, the pumps 110 flow fluid into the first and/or second cell culturing areas at intermittent intervals, such as once an hour, every day, every two days, once every three days, or once every week. In yet other implementations, the pumps 110 flow fresh or recirculated fluid through the first and/or second cell culturing areas continuously.

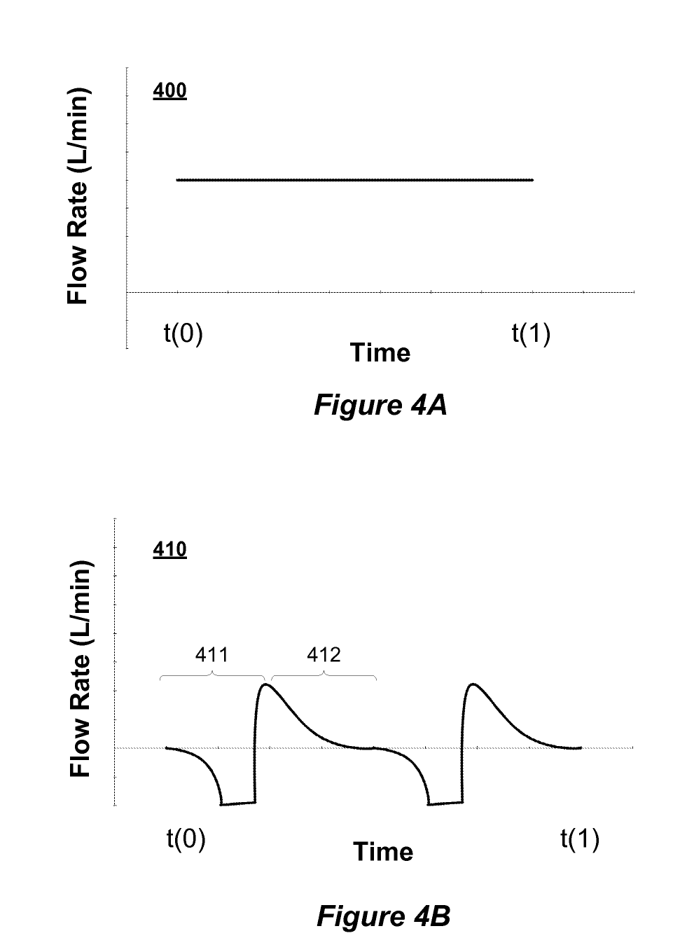

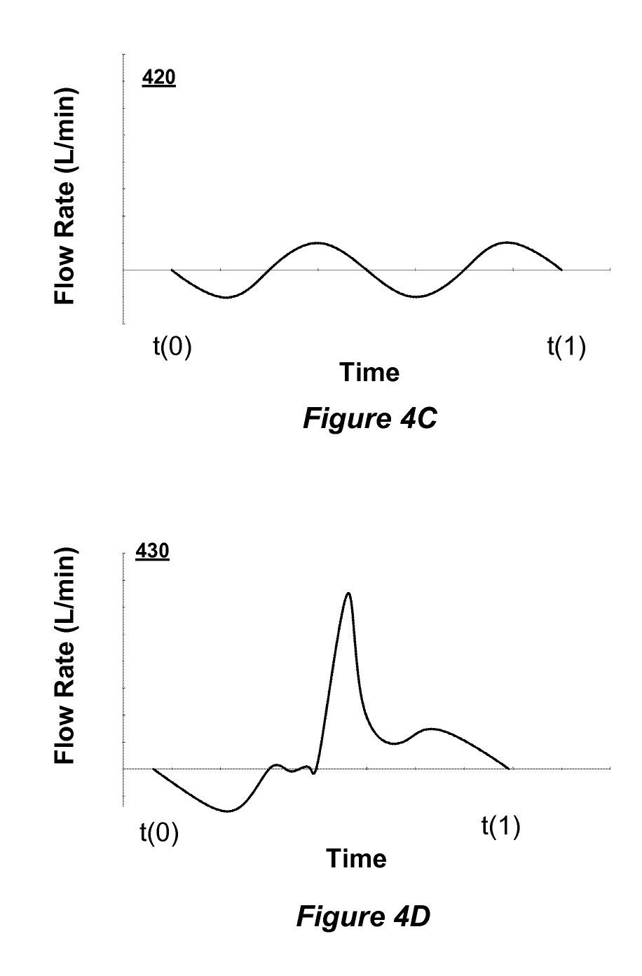

As described in greater detail in relation to FIG. 4, gas is flowed through the flow chamber 102. The flow of gas through the flow chamber 102 is managed by the gas pump 114. The gas pump 114 is configurable to flow gas through the flow chamber 102 in a substantially laminar, substantially non-laminar, continuous and/or pulsatile manner. The system 100 includes a plurality of gas reservoirs 116. The gas reservoirs 116 may store oxygen, carbon dioxide, nitrogen, argon, neon, methane, compressed air, or any combination thereof. In some implementations, the gas pump 114 receives gas from the one or more gas reservoirs 116 and/or ambient room air and mixes the gases before flowing the gas mixture through the flow chamber 102. In some implementations, the gas is mixed by a gas mixer prior to the gas pump 114 flowing the gas mixture through the flow chamber 102. In some implementations, the gas mixture that is flowed through the flow chamber 102 is substantially similar to the composition of gases that are exhaled by a human. For example, about 18% O₂, about 78% N₂, and about 4% CO₂. In some implementations, gases or gas mixtures that may affect transmissibility of a pathogen are introduced into the gas flowed through the flow chamber 102. For example, one or more aerosolized drugs or volatile organic compounds can be added to the gas flowed through the flow chamber 102.

When gas exits the flow chamber 102, it is collected by the collection system 118. The collection system 118 collects exhaust gas from the flow chamber 102 and filters the gas. In some implementations, the gas exiting the flow chamber 102 may include pathogens and/or other toxic (or dangerous) materials. The collection system 118 filters and substantially removes the undesirable materials from the exhaust gas before the gas is vented into the environment. In some implementations, the collection system 118 collects and quantifies the amount of pathogens and/or other toxic (or dangerous) materials are within the gas exiting the flow chamber 102. As described below, in some implementations, the gas pump 114 can drive gas through the flow chamber 102 in a first direction and then drive gas through the flow chamber 102 in the reverse direction. In these implementations, the collection system 118, houses the gas before it is returned through the flow chamber 102 during the reverse flow period.

In some implementations the flow chamber 102 allows for the visual inspection of the cells seeded within the cell culturing area (and other areas) of the flow chamber 102. For example, the top of the flow chamber 102 may include a glass cover slip through which the interior of the flow chamber 102 is viewable. The system 100 includes a microscope 120 to view the interior of the flow chamber 102. In some implementations, the microscope 120 is configured to record still or moving images of the cells within the flow chamber 102. In some implementations, the microscope 120 is an optical light microscope, confocal microscope, fluorescent microscope, or, in general, any type of microscope used in the field of cellular imaging and analysis. In some implementations, the microscope 120 is equipped with cellular analysis software that allows for the detection and/or classification of cells.

The system 100 also includes a control system 122. The control system 122 controls and manages the various above described components of the system 100. In some implementations, the control system 122 is a general computing device. In some implementations, the control system 122 includes one or more processors and at least one computer readable medium. Processor executable instructions are stored on the computer readable medium. When executed, the instructions cause the control system 122 to perform the control sequences described herein. For example, the control system 122 controls the flow of fluid into and out of the cell culture chambers by controlling the pumps 110. Similarly, the control system 122 can control the flow of gas through the flow chamber 102 via the gas pump 114. In some implementations, the control system 122 controls the pumps 110 and the gas pump 114 via an electrical connection. For example, the control system 122 may transmit a transistor—transistor logic (TTL) pulse, a pulse width modulated signal, or similar signal to the pumps 110 and/or gas pump 114 for control. In some implementations, the control system 122 is configured to control the state (On or Off) of the various pumps in the system 100. In some implementations, the control system 122 is configured to control the rate gases and/or fluid flows through the flow chamber 102. In some implementations, the control system 122 is configured to control the gas pump 114 such that the gas pump 114 flows gas through the flow chamber 102 at a rate of 30 L/min. In other implementations, the flow rate is between about 0 L/min and 20, between about 20 L/min and about 40 L/min, between about 40 L/min and about 60 L/min, between about 60 L/min and about 80 L/min, or between about 80 L/min and about 100 L/min. In some implementations, the shear stress is about 0.4-1.0 dynes/cm², about 1.0-2.0 dynes/cm², about 2.0-5.0 dynes/cm², about 5.0-10.0 dynes/cm². In models mimicking breathing conditions experienced during exercising or coughing, the shear stress may be between about 1000-1200 dynes/cm², about 1200-1400 dynes/cm², about 1400-1600 dynes/cm², about 1600-1800 dynes/cm², and about 1800-2000 dynes/cm².

The system 100 also includes a plurality of sensors 112 that are coupled to the control system 122. The sensors 112 are used by the control system 122 to monitor the conditions of the system 100. Example sensors 112 can include temperature sensors, humidity sensors, gas sensors, pH sensors, and sensors to monitor transepithelial electrical resistance. The sensors 112 are used to monitor the internal environment of the incubator 104 and/or flow chamber 102. In some implementations, the sensors 112 are flow sensors and monitor the flow of gas and liquid into and out of the flow chamber 102. The control system 122 uses the sensors 112 as feedback sensors for the various flow and control sequences described above. In some implementations, the data (e.g., temperature and flow rates) recorded with the sensors 112 is saved by the control system 112 for later analysis.

FIG. 2 illustrates a cross sectional view of one implementation of a flow channel 200 from the flow chamber 102 of the system 100. In some implementations, the flow chamber 102 is a microfluidic flow device configured to allow gas flow 218 across one or more cell cultures. As illustrated, the flow channel 200 includes a first cell culture chamber 202 positioned toward a proximal end of the flow chamber 200 and a second cell culture chamber 204 positioned toward a distal end of the flow chamber 200. The first cell culture chamber 202 is separated from the second cell culture chamber 204 by a barrier space 206. As illustrated, the barrier space 206 is coated with a barrier fluid 208. In each cell culture chamber, cells 210(a) and 210(b) (or collectively referred to as cells 210) sit atop a permeable membrane 212. The permeable membranes 212 (or similar support structure) allow molecules (e.g., nutrients in the growth medium) to pass from the fluid reservoirs 214 to the cells 210. The roof 216 of the flow chamber 102 is configured to allow visualization of the cells 210 within the flow chamber 102.

The flow chamber 102 is made from a material suitable for cell culture. In some implementations, the material is non-toxic to cells, durable enough to support the fluid in the fluid reservoirs 214 and the flow of gas through the flow chamber 102, and/or substantially non-reactive. For example, the flow chamber 102 can include polymeric and/or non-polymeric materials, including polydimethylsiloxane (PDMS), acrylic, polyethylene, polyolefin polymer, polyurethane, polystyrene, Pyrex, glass, polypropylene, Permanox, or any combination thereof. In some implementations, the flow chamber 102 is manufactured using photolithographic techniques, injection molding, direct micromachining, deep RIE etching, hot embossing, or any combinations thereof.

FIG. 2 illustrates one flow channel 200 of flow chamber 102, however, flow chamber 102 is not limited to having only one flow channel 200. In some implementations, flow chamber 102 includes a plurality of flow channels 200, each with a first cell culture chamber 202, second cell culture chamber 204, and barrier space 206. For example, the flow chamber 102 may include 1, 2, 4, 8, 12, 16, 20, or 32 flow channels 200.

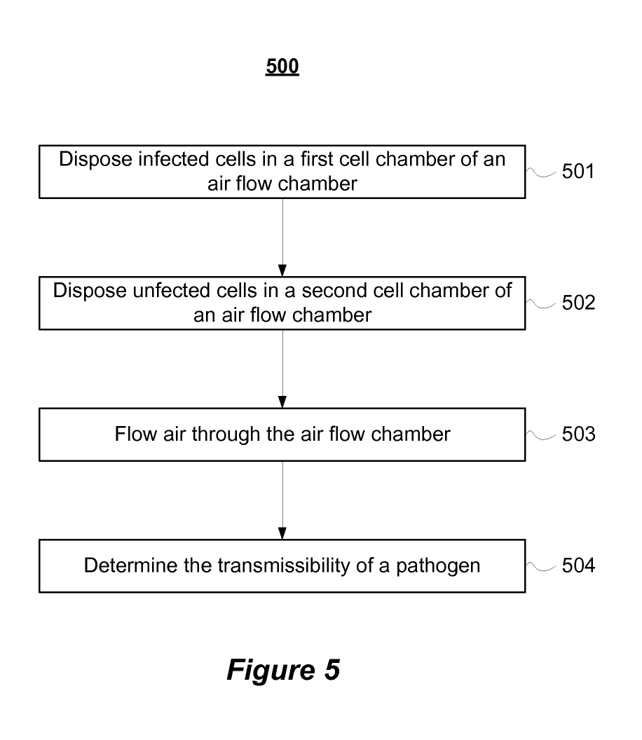

The first cell culture chamber 202 and second cell culture chamber 204 include a membrane 212. In some implementations, the cell culturing chambers 202 and 204 include cell culturing areas with a surface area of approximately 0.01-0.02 cm², 0.02-0.05 cm², 0.05-0.1 cm², 0.1-0.2 cm², 0.2-0.5 cm², 0.5-1 cm², 1-2 cm², 2-5 cm², 5-10 cm², or 10-25 cm². In some implementations, the membrane 212 thickness is between 10 μm and 500 μm. In some implementations, the membrane has a thickness between 100 nm and 500 μm. For instance, the membrane may be about 100-200 nm, about 200-500 nm, about 500 nm-1 μm, about 1-2 μm, about 2-5 μm, about 5-10 μm, about 10-20 μm, about 20-50 μm, about 50-100 μm, about 100-200 μm, or about 200-500 μm. The cells 210 and example methods for cell culturing are described below in relation to the method of FIG. 5 and the Examples.

The membrane 212 is compatible with cell metabolism and cell growth. The membrane 212 includes pores smaller than the diameter of the cells 210, but large enough to allow nutrients to pass from the reservoirs 214 to the cells 210. In some implementations, the cells 210 substantially adhere to the membrane 212, and create an air-liquid interface between the gas flowing through the flow channel 200 and the liquid in the fluid reservoir 214. In some implementations, the membrane 212 includes polycarbonate (PC), polyester (e.g., polyethylene terephthalate (PET)), collagen-coated polytetrafluoroethylene (PTFE), polydimethylsiloxane (PDMS), polysulfone, and natural electrospun ECM proteins (such as collagen), polycarbonates, polyesters, polytetrafluoroethylenes, ethylene-vinyl acetates (EVA), and polyvinyl acetates (PVA). In some implementations the membrane 212 is biodegradable and in some implementations the membrane 212 is non-biodegradable. In some implementations, at least a portion of the membrane 212 includes a topographic feature such as a plurality of grooves.

The length of the barrier space 206 can vary responsive to the type of pathogen tested. In some implementations, the length of the barrier space 206 is configured such that the gas flow 218 can carry the pathogen (or aerosolized particle) from the first cell culture chamber 202 to the second cell culture chamber 204. However, the length of the barrier space 206 is long enough that the pathogen cannot infect the cells 210(b) of the second cell culture chamber 204 in the absence of the gas flow 218. For example, the length of the barrier space 206 can be about 1-5 cm, about 5-10 cm, about 10-25 cm, about 25-50 cm, about 50-75 cm, or about 75-100 cm. In some implementations, the path of the barrier space 206 is tortuous. For example, and as described below in relation to FIGS. 3A and 3B, in some implementations the flow chamber 102 includes non-linear barrier spaces 206.

The top of the barrier space 206 is coated with a barrier fluid 208. In some implementations, only a sub-portion or none of the barrier space 206 is coated with the barrier fluid 208. In other implementations, substantially all of the barrier space 206 is coated with the barrier fluid 208. The barrier fluid 208 can be (or mimic by having similar characteristics) mucous or other airway fluids. For example, the barrier fluid 208 can include natural mucus (e.g., purified animal mucus) or artificial mucus synthesized from basic components. In other implementations, the barrier fluid 208 is, or simulates, mucus from the gut. In some implementations, the airway fluid 208 is produced by the cells 210. In some implementations, the barrier space 206 and/or other surfaces of the flow channel 200 are coated with enzymes, such as those used for ELISA assays, which can be used in the detection of a pathogen.

In some implementations, the flow channel 200 includes one or more actuators (not illustrated). The one or more actuators are configured to induce movement in the flow channel 200. For example, the one or more actuators may mimic the movement of the tracheal or alveolar basal membrane during a cough and/or normal respiration.

FIGS. 3A and 3B illustrate top views of the flow channels within the flow chamber 102 of FIG. 1. The flow channels 300 and 350 include a first cell culture chamber 202 separated from a second cell culture chamber 204 by a barrier space 302 and 304, respectively. As illustrated, the barrier space 302 is substantially linear (i.e., substantially straight). In contrast, the barrier space 304 is non-linear. In some implementations, the non-linear path of the barrier space 304 simulates greater distances between the first cell culture chamber 202 and second cell culture chamber 204 when compared to the linear barrier space 302. One of ordinary skill in the art will understand the flow chambers 300 and 350 are provided for illustrative purposes and are not intended to limit the scope of the disclosure to the illustrated barrier space designs.

FIGS. 4A-4D are plots of illustrative patterns the gas pump 114 can induce in the gas flow 218. In each gas flow pattern, the gas pump 114 flows gas through the flow chamber 102 from time t(0) until time t(1). A positive value for the gas flow pattern represents gas flowing from the inlet of the flow chamber 102 to the outlet of the flow chamber 102 (i.e., the gas is flowing from the gas pump 114 to the collection system 118). A negative value for the gas flow pattern represents gas flowing from the outlet of the flow chamber 102 to the inlet of the flow chamber 102 (i.e., the gas pump 114 is drawing gas back from the collection system 118 towards the gas pump 114).

In the gas flow pattern 400 of FIG. 4A, the gas pump 114 flows gas through the flow chamber 102 with a unidirectional, constant flow rate. Example flow rates may include any combination of the flow rates described above in relation to FIG. 1. In some implementations, the flow rate is selected to impart a specific shear rate on the cells. In FIG. 4B, the gas flow pattern 410 simulates a human breathing pattern. As illustrated, the gas flow pattern 410 includes bi-directional flow. During the “inhalation” phase 411, the gas pump simulates an inhalation by drawing gas from the outlet side of the flow chamber 102 to the inlet side of the flow chamber 102. During the “exhalation” phase 412 the gas pump 114 then drives the “inhaled” gas back through the flow chamber 102. The gas flow pattern 410 can include peak flow rates between about 1-15 L/min, about 15-30 L/min, about 30-45 L/min, about 45-60 L/min, about 60-75 L/min, and about 75-90 L/min.

The gas flow pattern 420, illustrated in FIG. 4C, is a sinusoidal flow pattern. The gas flow pattern 420 may include flow rates similar to those described above in relation to the gas flow pattern 410. In some implementations, the gas flow pattern 420 represents a simplified breathing pattern. In some implementations, the “inhalation” and “exhalation” phases of the gas flow pattern 420 may have varying or non-equal flow rates. One of ordinary skill in the art will recognize that other waveforms may be used with the flow chamber 102. For example, the gas pump 114 may flow square wave flow patterns, saw tooth wave flow patterns, non-continuous wave flow patterns, or any combination thereof through the flow chamber 102.

The gas flow pattern 430, illustrated in FIG. 4D, simulates a human cough. In gas flow patter 430, gas is slowly drawn into the flow chamber 102 from the collection system 118 (or outside source). The gas is then quickly flowed back through the flow chamber 102 in a flow pattern that simulates a cough. Peak flow rates during the gas flow pattern 430 can be about 100-200 L/min, about 200-300 L/min, about 300-400 L/min, or about 400-500 L/min. In some implementations, the “cough” flow pattern 430 lasts about 0.02-0.5 seconds, about 0.5-1 seconds, or about 1-3 seconds. In some implementations, the gas flow pattern 430 is combined with one or more of the above described gas flow patterns.

FIG. 5 is a flow chart illustrating a method 500 for determining the transmissibility of a pathogen. Briefly, the method 500 begins with the first set of cells being disposed in a first cell culture chamber (step 501) and a second set of cells begin disposed in a second cell culture chamber (step 502). Next, gas is flowed through the flow chamber (step 503). After a predetermined amount of time, the transmissibility of the pathogen is determined (step 504).

As set forth above, and referring to FIG. 2, the first set of cell are disposed in the first cell culture chamber 202 (step 501) and the second set of cells are disposed in the second cell culture chamber 204 (step 502). As described above, the cells are disposed on the membrane 212 within their respective cell culture chambers. In some implementations, the first set of cells 210(a) are infected with a pathogen. The infection of the first set of cells 210(a) can occur prior to or after the cells 210(a) are placed in the flow chamber 102. In some implementations, once disposed within the cell culturing chambers, the first and/or second set of cells are cultured within the flow chamber 102 for a predetermined amount of time prior to beginning any experimentation. For example, the cells 210 may be allowed to create a substantially intact epithelial layer on the membrane 212 before experimentation begins.

The cells disposed in the cell culture chambers can include human cells or cells from an animal model (e.g., swine or avian animal models). Example cells include tracheal epithelial cells, bronchial epithelial cells, nasal epithelial cells, alveolar cells, tracheal tissue, bronchial tissue, and/or nasal tissue. In other implementations, the cells are commercially-available cells grown for experimentation purposes. In some implementations, it is beneficial to know the transmissibility of a pathogen within a donor patient. Cells may be harvested from the donor patient and disposed in the flow chamber 102.

After the predetermined amount of culturing time has elapsed, gas is flowed through the flow chamber 102 (step 503). As described in relation to FIGS. 4A-4D, there exists a plurality of gas flow patterns that can be used to flow gas through the flow chamber 102. In some implementations, a barrier fluid 208 is applied to at least the barrier space 206 of the flow chamber 102 prior to the flowing of a gas through the flow chamber 102. In some implementations, the gas is flowed through the flow chamber 102 for a predetermined amount of time. For example, the gas may be flowed through the flow chamber 102 for about 15 min to about 30 min, about 30 min to about 1 hr, about 1 hr to about 3 hr, about 3 hr to about 12 hr, about 12 hr to 1 day, or about 1 day to about 3 days. In some implementations, the gas is flowed through the flow chamber 102 until a predetermined amount of the cells are infected. For example, the gas may be flowed through the flow chamber 102 until about 10%, 25%, 50%, 75%, or up to substantially 100% of the second set of cells 210(b) are infected. In some implementations, the first and/or second set of cells are cultured in the flow chamber 102 for a second predetermined amount of time prior to the determination of an infection rate of the second set of cells 210(b).

Next, the transmissibility of the pathogen is determined (step 504). The transmissibility of the pathogen may be quantified by determining the percent of cells 210(b) that are infected after a given amount of time (or similarly the amount of cells 210(b) not infected after a given amount of time). For example, a pathogen that infects 75% of cells 210(b) after 1 day in the flow chamber 102 is more transmissible than a pathogen that infects 10% of cells 210(b) after 1 day in a second flow chamber 102. In other implementations, the transmissibility of the pathogen is quantified by the length of time it takes to infect a give percentage of the 210(b) cells. For example, using the flow chamber 102, it may be determined that a first pathogen infects 50% of the cells 210(b) after 1 day of exposure to the cells 210(a) and a second pathogen infects 87% of the cells 210(b) after 1 day of exposure to the cells 210(a). As described above, test agents, such as antibiotics, antivirals, antimicrobial compounds, antibodies, other cells, and/or mucus-modifying agents may be added to the nutrients provided to the cells 210(a) and/or the cells 210(b). In such an implementation, the above experiments are repeated, but with the test agent provided to the cells 210(b). Comparing the infection rate seen in the first and second experiments, the effectiveness of the antibiotic or other agent in preventing infection may be assessed.

Referring back to the step 504, in some implementations, the infection rate of the second set of cells 210(b) is determined when the cells are still housed in the flow chamber 102. For example, the cells of the second cell culture chamber 204 may express a fluorescence protein when exposed to the pathogen. The fluorescence can then be detected with the microscope 120. In other implementations, the second cell culture chamber 204 and/or the cells 210(b) are removed from the flow chamber 102 prior to a viral load being detected in the cells 210(b). The presence of the virus in the cells 210(b) can be detected with real-time PCR (qPCR), Northern blots, Southern blots, ELISA assays or similar detection methods.

### EXAMPLES

The Influenza strain is one example pathogen that may be used with the systems and methods described herein. Influenza strains are able to effectively replicate in the ciliated epithelium of the conducting airways of humans. Human-adapted strains bind with highest affinity to glycans of a specific topology that are terminated by sialic acid (SA) that is α2-6 linked to the penultimate galactose (or Gal) sugar (known as α2-6 sialylated glycans). α2-6 sialylated glycans are mostly present in the human upper respiratory epithelia. Deep lung epithelia of the human alveolae predominantly express α2-3 sialylated glycans. Since transformed cell lines express a multitude of surface glycan receptors, including those with α2-3 linked SA and α2-6 linked SA, they are generally capable of supporting replication of a wide variety of influenza strains. While useful for replicating influenza in vitro and studying certain aspects of the infection process, transformed cell lines do not truly represent the viral-host interactions that occur in the human respiratory tract.

Primary human tracheal or bronchial epithelial cells grown on a membrane support with an air-liquid interface (ALI) differentiate into a pseudostratified epithelium with morphological and physiological features representative of the human conducting airway in vivo. For example, the cells differentiate into a heterogenous population of ciliated and non-ciliated cells (e.g., secretory, goblet/mucous-producing, and ciliated basal), which create a more representative model to study respiratory virus infection. In some implementations with human tracheobronchial epithelial cells grown with an ALI, ciliated cells are preferentially infected by avian-adapted influenza strains while human-adapted strains prefer to target non-ciliated cells.

To test the transmissibility of influenza strains (e.g., influenza strains PR/8, Brisbane/10 and Brisbane/59), primary normal human bronchial epithelial (NHBE) cells are seeded onto the membrane 212 in the first cell culture chamber 202 for at least 21 days in preparation for viral infection studies. In some implementations, a 21-day culture achieves sufficient differentiation; though longer times of differentiation, up to 35 days, may be used. Immunohistochemistry (IHC) of markers of NHBE differentiation are used to identify goblet/mucosa cells (Muc5AC), ciliated cells (Foxj1) and basal cells (CK5).

In general, the cell culture protocol includes expanding frozen cell stock in a T75 flasks using Lonza growth medium. The cell culture chamber is coated with collagen, and the cells are seeded into the cell culture chambers (50K per cell culture chamber). The cells are allowed to adhere and acclimate to the cell culture chamber in growth medium for three days at 37° C. and 5% CO₂. After three days the growth medium is removed from the apical and basal side of the membrane. The apical side corresponds to the portion of the cell culture chamber above the membrane 212 and the basal side corresponds to the fluid reservoirs 214 below the membrane 212. A differentiation medium is the added to the basal side (fluid reservoir 214) of the cell culture chambers. The cells are then allowed to differentiate for about 21 to about 35 days. The pump 110 exchanges the medium in the fluid reservoir 214 every two days. On average, the cells start producing mucus around day 13. During the differentiation period, the apical surface is washed with sterile PBS once per week.

The process of infecting the cells in the first cell culture chamber 202 begins once the cells are suitably differentiated (e.g., a differentiated epithelial monolayer has formed across the membrane 212). First, the apical surface is washed three times with PBS. An influenza strain is mixed with 100 μl of PBS and added to the apical surface of the first cell culture chamber. An example MOI for the virus is between about 0.001 to 0.1. The cells are then incubated at 37° C. for 1 hour. The fluid is then aspirated from the apical surface and the cells are washed with 100 μl PBS. The cells were returned to the incubator for about 24 hr to 72 hr.

After the cells of the first cell culture chamber are infected, the flow chamber is placed in the system described herein. Gas is flowed through the flow chamber 102 as described above in relation to FIGS. 4A-4D. After a predetermined flow period, the cells are removed from the flow chamber, and in some implementations allowed to further incubate for 24 hr to 72 hr. The viral load is quantified by plaque assay on the MDCK cells or by qPCR.

The invention may be embodied in other specific forms without departing from the spirit or essential characteristics thereof. The forgoing implementations are therefore to be considered in all respects illustrative, rather than limiting of the invention.

What is claimed:

1. A method of determining transmissibility of a pathogen, the method comprising:
disposing at least one cell infected with a pathogen in a first cell chamber disposed toward a proximal end of a gas flow channel;
disposing at least one uninfected cell in a second cell chamber disposed toward a distal end of the gas flow channel, the second cell chamber separated from the first cell chamber by a barrier space;
flowing gas through the gas flow channel such that the gas flows across the at least one infected cell and then flows across the at least one uninfected cell; and
determining an infection rate of the uninfected cells.

2. The method of claim 1, wherein flowing the gas through the gas flow channel comprises flowing gas through the gas flow channel in a pulsatile manner.

3. The method of claim 1, wherein flowing the gas through the gas flow channel comprises flowing gas through the gas flow channel in a matter that simulates a cough.

4. The method of claim 1, further comprising incubating the at least one uninfected cells for a predetermined amount of time before determining the infection rate of the uninfected cells.

5. The method of claim 1, wherein determining the infection rate further comprises determining a transmissibility of the pathogen.

6. The method of claim 1, wherein determining the infection rate further comprises measuring a viral load of the at least one uninfected cell.

7. The method of claim 1, further comprising introducing a fluorescent protein into the at least one uninfected cell, wherein the fluorescent protein fluoresces when the at least one uninfected cell is infected by the pathogen from the at least one infected cell.

8. The method of claim 7, wherein determining the infection rate further comprises measuring a fluorescence level.

9. The method of claim 1, wherein the pathogen is a virus.

10. The method of claim 9, wherein the virus is an influenza virus.

11. The method of claim 1, further comprising applying an airway fluid to at least one wall of the barrier space.

12. The method of claim 1, further comprising measuring at least one condition in the gas flow channel.

13. The method of claim 12, wherein the condition is one of a temperature, a pressure, a flow rate, and a humidity.

14. A system for determining the transmissibility of a pathogen, the system comprising:
a gas flow channel having a proximal end and a distal end, the gas flow channel further comprising:
a first cell chamber disposed toward the proximal end of the gas flow channel, the first cell chamber configured to supply nutrients to a basolateral surface of at least one first cell;
a second cell chamber disposed toward the distal end of the gas flow channel, the second cell chamber configured to supply nutrients to a basolateral surface of at least one second cell; and
a barrier space between the first cell chamber and the second cell chamber, the barrier space configured to allow an aerosolized particle to pass from the first cell chamber to the second cell chamber; and
a gas pump configured to flow gas through the gas flow channel at a predetermined flow rate; and
an incubator configured to maintain a predetermined atmospheric condition within the gas flow channel.

15. The system of claim 14, wherein the gas pump is configured to flow gas through the gas flow channel in a manner that simulates a cough.

16. A flow device comprising:
a gas flow channel having a proximal end and a distal end, the gas flow channel further comprising:
a first cell chamber disposed toward the proximal end of the gas flow channel, the first cell chamber configured to supply nutrients to a basolateral surface of at least one first cell;
a second cell chamber disposed toward the distal end of the gas flow channel, the second cell chamber configured to supply nutrients to a basolateral surface of at least one second cell; and
a barrier space between the first cell chamber and the second cell chamber, the barrier space configured to allow an aerosolized particle to pass from the first cell chamber to the second cell chamber.

17. The flow device of claim 16, wherein the first cell chamber and the second cell chamber comprise a permeable membrane disposed atop cellular wells.

18. The flow device of claim 16, wherein barrier space is non-linear.

19. The flow device of claim 16, wherein at least one surface of the barrier space is coated with an airway fluid.

20. The flow device of claim 16, wherein the gas flow channel is configured to induce a predetermined shear force on the at least one first cell and the at least one second cell.

21. The flow device of claim 19, wherein the airway fluid is a mucous.
